(19) United States
(12) Patent Application Publication
(10) Pub. No.: US 2024/0366957 A1
(43) Pub. Date: Nov. 7, 2024

(54) CUSTOMIZABLE TANNING APPARATUS

(71) Applicant: Randy MERSKY, Silver Spring, MD (US)

(72) Inventor: Randy MERSKY, Silver Spring, MD (US)

(21) Appl. No.: 18/143,754

(22) Filed: May 5, 2023

(51) Int. Cl. A61N 5/06

(52) U.S. Cl. CPC A61N 5/0614; A61N 2005/0632; A61N 2005/0638; A61N 2005/0667

(57) ABSTRACT

A tanning apparatus, including a support having a bottom and a top, a mount connected to the top of the support and configured to extend laterally from the support, and at least one light filter. The mount is operable to releasably hold the at least one light filter. The mount is also adjustably-sizable and is operable to accommodate the at least one light filter.

### BACKGROUND OF THE DISCLOSURE

### 1. Field of the Disclosure

The present disclosure relates to the field of outdoor tanning or sunbathing devices. More particularly, the present disclosure relates to a tanning apparatus configured to control multiple tanning zones on the body.

### 2. Background of the Disclosure

The current ways to manage tanning outdoors and to protect the skin from adverse exposures include lotions, sprays, protective clothing, umbrellas and/or other canopy type devices. Each of these, however, have problems/limitations and do not effectively allow for the ability to selectively control multiple tanning zones and sun exposure.

For example, people forget to use, don't bother with, or sometimes run out of sunscreen. Additionally, sunscreen lotions or sprays are messy and can wash off (or sweat off) in varying degrees, resulting in uneven or inconsistent tans as well as an increased chance of sunburn. Furthermore, sunscreen lotions need to be reapplied often and/or are often not evenly applied. Sunscreen lotions are also not easily applied to some parts of the body (for example, back, top of the head, e.g., with thinning hair). This further results in an increased likelihood of an uneven tan or sunburn. Also, sunscreen lotions come in various SPF numbers that are not clear as to the correlation of skin protection. This further reduces the chance of achieving a desired tan level.

For example, umbrellas, canopies, and/or tents in all sizes and shapes to block the body from sunlight. These include chair canopies, tents, umbrellas, etc. Most, if not all umbrellas, canopies, and/or tents, are produced with a type of decorative fabric material with (e.g., UPF+50) and primarily serve as shade producers or total tanning blockers for either the entire body or some portion of the body. Umbrellas, canopies, and tents do not allow for variable tanning options and are often an all or nothing approach to UV sun exposure. Often the user is either in the dense shade of the umbrella, canopy, and/or tent, or exposed to the sun. Additionally, umbrellas, canopies, and tents can be inconvenient since two or more people often attempt to share one covering. This may leave at least one person at least partially uncovered resulting in uneven or inconsistent UV or sun protection. Also, people often move in and out of the shade of canopies based on cloud covering and/or temperature, which can also result in inconsistent UV protection.

Various clothing (e.g., hats, shirts, glasses, pants) only cover a small part of the body and normally totally block sun exposure. Therefore, some portions of the body are fully exposed to the sun while other parts or portions of the body are completely blocked from sun exposure leading to uneven tanning or tan lines. Additionally, clothing can be uncomfortable or hot to wear causing the wearer to adjust or take off the protective clothing and further result in inconsistent or uneven tanning.

In view of the above, a need in the art has arisen for a device that has the ability to control multi-tanning zones on the body by controlling the degree of skin exposure.

### SUMMARY OF THE EMBODIMENTS OF THE DISCLOSURE

The novel features, which are characteristic of the disclosure, both as to structure and method of operation thereof, together with further aims and advantages thereof, will be understood from the following description, considered in connection with the accompanying drawings, in which embodiments of the disclosure are illustrated by way of example. It is to be expressly understood, however, that the drawings are for the purpose of illustration and description only, and they are not intended as a definition of the limits of the disclosure.

Aspects of the disclosure are directed to a tanning apparatus including a support having a bottom and a top, a mount connected to the top of the support and configured to extend laterally from the support, and at least one light filter. The mount is operable to releasably hold the at least one light filter. The mount is also adjustably-sizable and is operable to accommodate the at least one light filter.

In further embodiments of the disclosure, the mount includes a longitudinally-extending frame connected to the top of the support and extending in a longitudinal direction, the frame having a first end and a second end.

In embodiments, the adjustably-sizable mount includes at least one arm connected to and extending from at least one end of the longitudinally-extending frame in a transverse direction approximately perpendicular to the longitudinal direction.

In further embodiments, the at least one arm is configured to increase in overall length in the transverse direction to accommodate at least one additional light filter.

In embodiments, the adjustably-sizable mount includes a second longitudinally-extending frame connected to the at least one arm and extending in the longitudinal direction.

In additional embodiments, the adjustably-sizable mount may include a first arm connected to and extending from the first end of the longitudinally-extending frame in a transverse direction approximately perpendicular to the longitudinal direction.

In further embodiments, the adjustably-sizable mount may also include a second arm connected to and extending from the second end of the longitudinally-extending frame in the transverse direction approximately perpendicular to the longitudinal direction.

In even further embodiments, the adjustably-sizable mount may also include a second longitudinally-extending frame connected to the first arm and extending in the longitudinal direction.

In other embodiments, the adjustably-sizable mount may additionally include a second arm connected to and extending from the second end of the frame in the transverse direction approximately perpendicular to the longitudinal direction while the second arm is also connected to the second longitudinally-extending frame.

In additional embodiments, the longitudinally-extending frame is configured to increase in overall length in the longitudinal direction to accommodate at least one additional light filter.

In some embodiments, the adjustably-sizable mount includes at least one telescoping member.

In other embodiments, the adjustably-sizable mount includes at least one folding member.

In other embodiments, the adjustably-sizable mount includes at least two separate pieces assembleable together.

In additional embodiments, the first arm is configured to increase in overall length in the transverse direction to accommodate at least one additional light filter.

In some embodiments, the first arm is a telescoping member.

In other embodiments, the first arm is a folding member.

In embodiments, the first arm is configured to increase in overall length in the transverse direction and the longitudinally-extending frame is configured to increase in overall length in the longitudinal direction to each accommodate at least one additional light filter.

In further embodiments, the tanning apparatus also includes a joint arranged between the mount and the support, the joint enabling a relative pivoting movement between the mount and the support.

In additional embodiments, the support is a single support extending from the bottom to the top in an approximately vertical direction of the tanning apparatus.

In embodiments, the support may be configured to be removably embedded in ground.

In other embodiments, the tanning apparatus also includes a base, and the support is configured to removably attach to the base.

In other embodiments, the tanning apparatus also includes a controller, wherein the base is operable to rotate the support, and the controller is configured to control position of the support.

In other embodiments, the tanning apparatus also includes a sensor, wherein the controller is configured to control rotation of the support in response to a detection by the sensor.

In embodiments, the tanning apparatus also includes a controller, wherein the support is operable to rotate the mount, and the controller is configured to control position of the mount.

In some embodiments, the tanning apparatus also includes a controller and a sensor, with the base being able to rotate the support, and the controller may be configured to rotate the support based on a current sun position detected by the sensor.

In embodiments, the at least one filter includes multiple filters supported along the mount, wherein at least two of the filters have different filtering properties from each other.

In additional embodiments, the tanning apparatus may include a plurality of joints, and each of the plurality of joints is configured to selectively connect two of the plurality of filters to each other, or one of the plurality of filters to the mount.

In further embodiments, the plurality of joints enables at least one of rotation of each of the at least two filters relative to the mount and/or rotation of one of the at least two filters relative to another of the at least two filters to stack filters.

In additional embodiments, the at least one filter of the tanning apparatus includes at least three filters configured to be arranged serially along a longitudinal direction of the mount, with at least two of the filters having different filtering properties from each other.

In embodiments, the at least one filter comprises a plurality of filters configured to be interchangeable with each other.

In some embodiments, the mount may be generally rectangular.

In additional embodiments, the support includes a telescoping pole arrangeable in an approximately vertical direction and configured to selectively extend and retract in the approximately vertical direction.

In embodiments, the at least one filter comprises at least one of glass and/or plastic.

In further embodiments, the at least one filter is a photochromatic filter, an electrochromatic filter, or a filter having fixed filtering properties.

In other embodiments, the at least one filter includes a user-selectable film tinting actuable by a controller configured to control the user-selectable film tinting based on a selected tanning preference of a user.

In some embodiments, the at least one filter is a planar filter. Additionally, the at least one filter may be a rigid filter.

In further embodiments, the at least one filter is a planar filter or a roll filter, and the mount is configured to support each of the planar filter and the roll filter.

In even further embodiments, the mount is configured to concurrently support each of the planar filter and the roll filter.

In embodiments, the at least one filter is a roll filter. The roll filter in an un-deployed orientation is unrollable to position the roll filter on the mount in a deployed orientation.

In additional embodiments, the roll filter includes different filter regions along a length of the roll filter, at least two of the different filter regions having differing light filtering properties from each other.

In other embodiments, the tanning apparatus also includes a second roll filter, the roll filter in the un-deployed orientation is supportable on a first end of the mount, and the second roll filter in the un-deployed orientation is supportable on a second end of the mount.

In some embodiments, the support includes at least one pole extending from the bottom to the top in an approximately vertical direction.

In some embodiments, the support is attached at mid-way point along the mount.

Additional, aspects of the disclosure are directed to a tanning apparatus including a support having a bottom and a top, a mount connected to the top of the support and configured to extend laterally from the support, and a plurality of light filters. The mount may be operable to releasably hold the plurality of light filters, and at least two of the plurality of light filters have different filtering properties from each other.

In some embodiments, the mount is an adjustably-sizable mount.

In some embodiments, the tanning apparatus further includes a plurality of joints, wherein each of the plurality of joints is configured to selectively connect two of the plurality of light filters to each other, or one of the plurality of light filters to the mount.

In additional embodiments, the support includes a telescoping pole arrangeable in an approximately vertical direction and configured to selectively extend and retract in the approximately vertical direction.

In other embodiments, the plurality of light filters includes a user-selectable film tinting actuable by a controller configured to control the user-selectable film tinting based on a user selection.

In further embodiments, the plurality of light filters are at least one of a planar filter or a roll filter, and the mount is configured to support each of the planar filter and the roll filter.

In further embodiments, the mount is configured to concurrently support each of the planar filter and the roll filter.

In additional embodiments, the plurality of light filters include at least one roll filter, wherein the roll filter in an un-deployed orientation is unrollable to position the roll filter on the mount in a deployed orientation.

In embodiments, the roll filter includes different filter regions along a length of the roll filter, at least two of the different filter regions having differing light filtering properties from each other.

In some embodiments, the tanning apparatus further includes a second roll filter, wherein the roll filter in the un-deployed orientation is supportable on a first end of the mount, and the second roll filter in the un-deployed orientation is supportable on a second end of the mount.

In additional embodiments, the plurality of light filters include at least one of glass and/or plastic.

In some embodiments, at least one of the plurality of light filters is a photochromatic filter.

In embodiments, at least one of the plurality of light filters is an electrochromatic filter.

In additional embodiments, at least one of the plurality of light filters is a filter having fixed filtering properties.

In embodiments, the plurality of light filters are planar filters.

In additional embodiments, the plurality of light filters are rigid filters.

In embodiments, the plurality of light filters include at least three filters configured to be arranged serially along a longitudinal direction of the mount, wherein at least two of the at least three filters have different filtering properties from each other.

In further embodiments of the disclosure, the mount includes a longitudinally-extending frame connected to the top of the support and extending in a longitudinal direction, the frame having a first end and a second end.

In additional embodiments, the mount may include a first arm connected to and extending from the first end of the longitudinally-extending frame in a transverse direction approximately perpendicular to the longitudinal direction.

In further embodiments, the mount may also include a second arm connected to and extending from the second end of the longitudinally-extending frame in the transverse direction approximately perpendicular to the longitudinal direction.

In even further embodiments, the mount may also include a second longitudinally-extending frame connected to the first arm and extending in the longitudinal direction.

In other embodiments, the mount may additionally include a second arm connected to and extending from the second end of the frame in the transverse direction approximately perpendicular to the longitudinal direction while the second arm is also connected to the second longitudinally-extending frame.

In additional embodiments, the longitudinally-extending frame is configured to increase in overall length in the longitudinal direction to accommodate at least one additional light filter.

In some embodiments, the longitudinally-extending frame is a telescoping member.

In other embodiments, the longitudinally-extending frame is a folding member.

In additional embodiments, the first arm is configured to increase in overall length in the transverse direction to accommodate at least one additional light filter.

In some embodiments, the first arm is a telescoping member.

In other embodiments, the first arm is a folding member.

In embodiments, the first arm is configured to increase in overall length in the transverse direction and the longitudinally-extending frame is configured to increase in overall length in the longitudinal direction to each accommodate at least one additional light filter.

In further embodiments, the tanning apparatus also includes a joint arranged between the mount and the support, the joint enabling a relative pivoting movement between the mount and the support.

In additional embodiments, the support is a single support extending from the bottom to the top in an approximately vertical direction of the tanning apparatus.

In embodiments, the support may be configured to be removably embedded in ground.

In other embodiments, the tanning apparatus also includes a base, and the support is configured to removably attach to the base.

In some embodiments, the tanning apparatus also includes a controller and a sensor, with the base being able to rotate the support, and the controller may be configured to rotate the support in response to current sun position detected by the sensor.

In further embodiments, the plurality of joints enables at least one of rotation of each of the at least two filters relative to the mount and/or rotation of one of the at least two filters relative to another of the at least two filters to stack filters.

In embodiments, the plurality of filters are configured to be interchangeable with each other.

In some embodiments, the mount may be generally rectangular.

In some embodiments, the support includes at least one pole extending from the bottom to the top in an approximately vertical direction.

In some embodiments, the support is attached at mid-way point along the mount.

Additional, aspects of the disclosure are directed to a tanning apparatus including a support having a bottom and a top, a base releasably attachable to the bottom of the support, a mount connected to the top of the support and configured to extend laterally from the support, and at least one light filter. The mount may be operable to releasably hold the at least one light filter and the mount is configured to adjustably pivot relative to the support. Also, the support may vertically adjust.

In some embodiments, the support is attached at mid-way point along the mount.

Other exemplary embodiments and advantages of the present disclosure may be ascertained by reviewing the present disclosure and the accompanying drawings, and the above description should not be considered to limit the scope of the present disclosure.

### BRIEF DESCRIPTION OF THE DRAWINGS

The present disclosure is further described in the detailed description which follows, in reference to the noted plurality of drawings, by way of non-limiting examples of preferred embodiments of the present disclosure, in which like characters represent like elements throughout the several views of the drawings, and wherein:

FIG. 1 shows a perspective view of an exemplary tanning apparatus with two filters in accordance with aspects of the disclosure;

FIG. 2a shows a front view of an exemplary tanning apparatus with the mount in a position approximately perpendicular to the support in accordance with aspects of the disclosure;

FIG. 2b shows a front view of an exemplary tanning apparatus with the mount in a raised vertical position in accordance with aspects of the disclosure;

FIG. 3a shows a front view of an exemplary tanning apparatus with the support and the support extension in an un-extended position in accordance with aspects of the disclosure;

FIG. 3b shows a front view of an exemplary tanning apparatus with the support and the support extension in an extended position in accordance with aspects of the disclosure;

FIG. 3c shows a front view of an exemplary tanning apparatus with a first support member and a second support member in an un-attached configuration in accordance with aspects of the disclosure;

FIG. 3d shows a front view of an exemplary tanning apparatus with the first support member and the second support member in an attached configuration in accordance with aspects of the disclosure;

FIG. 4a shows a front view of an exemplary tanning apparatus with a base in accordance with aspects of the disclosure;

FIG. 4b shows a front view of an exemplary tanning apparatus with a stake in accordance with aspects of the disclosure;

FIG. 4c shows a perspective view of an exemplary tanning apparatus with support fasteners which may be attached to the base and/or the support in accordance with aspects of the disclosure;

FIG. 5a shows a top view of an exemplary tanning apparatus with a single filter in accordance with aspects of the disclosure;

FIG. 5b shows a top view of an exemplary tanning apparatus with four filters in accordance with aspects of the disclosure;

FIG. 5c shows a top view of an exemplary tanning apparatus with six filters in accordance with aspects of the disclosure;

FIG. 6a shows a perspective view of an exemplary tanning apparatus with two supports in accordance with aspects of the disclosure;

FIG. 6b shows a perspective view of an exemplary tanning apparatus with three supports in accordance with aspects of the disclosure;

FIG. 6c shows a perspective view of an exemplary tanning apparatus with four supports in accordance with aspects of the disclosure;

FIG. 7a shows a top view of an exemplary tanning apparatus with a mount with a single frame in accordance with aspects of the disclosure;

FIG. 7b shows a top view of an exemplary tanning apparatus with a mount in an L-shape configuration in accordance with aspects of the disclosure;

FIG. 7c shows a top view of an exemplary tanning apparatus with a mount in a C-shape configuration in accordance with aspects of the disclosure;

FIG. 7d shows a top view of an exemplary tanning apparatus with a mount in a U-shape configuration in accordance with aspects of the disclosure;

FIG. 7e shows a top view of an exemplary tanning apparatus with a mount in a rectangular configuration in accordance with aspects of the disclosure;

FIG. 7f shows a perspective view of an exemplary tanning apparatus with a tent-like configuration;

FIG. 8a shows a top view of an exemplary filter with a fixture in accordance with aspects of the disclosure;

FIG. 8b shows a perspective view of an exemplary mount with channels for attaching an exemplary filter with ridges in accordance with aspects of the disclosure;

FIG. 8c shows a perspective view of an exemplary mount with slide channels for sliding in an exemplary filter in accordance with aspects of the disclosure;

FIG. 9a is an exemplary tanning apparatus with a smart-film filter in accordance with aspects of the disclosure;

FIG. 9b is an exemplary tanning apparatus with a smart-film filter in accordance with aspects of the disclosure;

FIG. 10 is an exemplary tanning apparatus with a smart base in accordance with aspects of the disclosure;

FIG. 11 shows a top view of an exemplary filter with multiple filter regions in accordance with aspects of the disclosure;

FIG. 12a shows a top view of an exemplary tanning apparatus with filters connected with joints and positioned approximately perpendicular with respect to the mount in accordance with aspects of the disclosure;

FIG. 12b shows a top view of an exemplary tanning apparatus with the second filter pivoted up approximately parallel with respect to the first filter in accordance with aspects of the disclosure;

FIG. 12c shows a top view of an exemplary tanning apparatus with a first filter and a second filter pivoted approximately at a 45-degree angle with respect to the frame in accordance with aspects of the disclosure;

FIG. 13a shows a top view of an exemplary tanning apparatus with a roll filter in accordance with aspects of the disclosure;

FIG. 13b shows a top view of an exemplary tanning apparatus with a first roll filter and a second roll filter in accordance with aspects of the disclosure;

FIG. 13c shows a front view of an exemplary tanning apparatus with a roll filter in accordance with aspects of the disclosure;

FIG. 13d shows a front view of an exemplary tanning apparatus with a first roll filter and a second roll filter in accordance with aspects of the disclosure;

FIG. 14a shows a top view of an exemplary tanning apparatus with a roll filter and planar filter in accordance with aspects of the disclosure;

FIG. 14b shows a front view of an exemplary tanning apparatus with a roll filter and a planar filter in accordance with aspects of the disclosure;

FIG. 15 shows a top view of an exemplary roll filter with multiple filter regions in accordance with aspects of the disclosure; and

FIG. 16 shows an exemplary environment for practicing aspects of the present disclosure.

### DETAILED DESCRIPTION OF THE EMBODIMENTS OF THE DISCLOSURE

In view of the foregoing, the present disclosure through one or more of its various aspects, embodiments and/or specific features or sub-components, is thus intended to bring out one or more of the advantages as specifically noted below.

The particulars shown herein are by way of example and for purposes of illustrative discussion of the embodiments of the present disclosure only and are presented in the cause of providing what is believed to be the most useful and readily understood description of the principles and conceptual aspects of the present disclosure. In this regard, no attempt is made to show structural details of the present disclosure in more detail than is necessary for the fundamental understanding of the present disclosure, the description taken with the drawings making apparent to those skilled in the art how the several forms of the present disclosure may be embodied in practice.

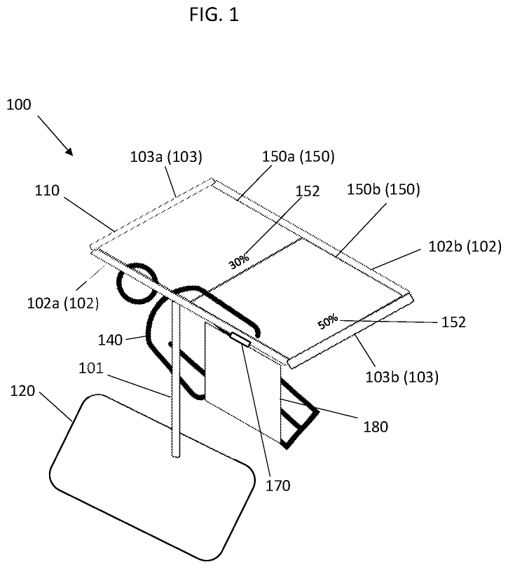

Referring now to the drawings wherein like characters represent like elements, FIG. 1 shows a perspective view of an exemplary tanning apparatus 100 with two filters 150a and 150b, in accordance with aspects of the disclosure. As shown in FIG. 1, the tanning apparatus 100 may include a support 101, a base 120, a mount 110, a shade 180, a shade fastener 170, a first filter 150a, and a second filter 150b. The filters 150a and 150b may include a label 152, as shown in FIG. 1, identifying one or more properties of each filter 150. Additionally, with this exemplary embodiment, the mount 110 includes a first frame 102a, a second frame 102b, a first arm 103a, and a second arm 103b. The first frame 102a, the second frame 102b, the first arm 103a, and the second arm 103b may be connected to form a substantially rectangular shape, as shown. In embodiments, the mount 110 may be connected or fastened to the top portion of the support 101. The one or more filters 150 may selectively attach or connect to the mount 110. The one or more filters 150 may also selectively filter or enhance light, for example, sunlight for tanning. The mount 110, including the frames 102 and the arms 103, may be generally adjustable and/or configured to extend out approximately perpendicular with respect to the support 101. The mount 110 may be generally parallel with the ground so that a user 140 may sit or lay beneath the filters 150 of the tanning apparatus 100.

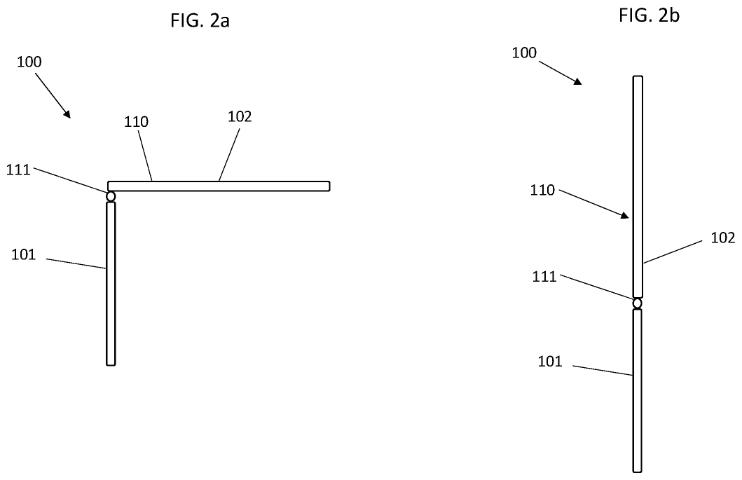

FIG. 2a shows a front view of an exemplary tanning apparatus 100 with the mount 110 in a position approximately perpendicular to the support 101 in accordance with aspects of the disclosure. As shown in FIG. 2a, the mount 110 may be fastened to the support 101 by a pivoting arrangement 111 (e.g., pivot, hinge, or swivel joint) to allow the mount 110 to selectively rotate, tilt, and/or pivot about the support 101. The pivoting arrangement 111 may be adapted for selective rotation of the mount 110 relative to the support 101 for setting a position thereof. The pivoting arrangement 111 may be, for example, a locking joint, a ratcheting joint, a locking hinge, or a locking hinged joint, which may be selectively locked in any desired position. For example, the pivoting arrangement 111 may lock to secure the mount 110 in an approximately perpendicular position, with respect to the support 101, as shown in FIG. 2a. Additionally, the pivoting arrangement 111 may be selectively unlocked from the approximately perpendicular position and pivoted to and locked in any desired position (e.g., to adjust for the moving sun). The approximately perpendicular position of the mount 110, as shown in FIG. 2a, demonstrates where the mount 110 may provide cover or act as a barrier between the sunlight and the user. For example, the mount 110 can rotate and/or tilt on the support 101 to adjust the amount of cover and/or UV protection provided by the filters 150 depending on the position and/or movement of the sun.

FIG. 2b shows a front view of an exemplary tanning apparatus 100 with the mount 110 in a raised vertical position in accordance with aspects of the disclosure. As shown in FIG. 2b, the pivoting arrangement 111 may be locked to secure the mount 110 in an approximately parallel position with respect to and aligned with the support 101. The approximately vertical position, as shown in FIG. 2b, may provide the user easier access underneath the apparatus 100. In accordance with aspects of the disclosure, when the mount is in the approximately vertical position, as shown in FIG. 2b, the user can avoid contact with the mount 110 when standing up or sitting down.

FIG. 2a and FIG. 2b, respectively, show the mount 110 in a substantially perpendicular position to the support 101 and a substantially vertical position. It should be understood, however, these angles/positions are non-limiting and exemplary, and the mount 110 may be pivoted and locked into a position at any angle. For example, the mount 110 may adjust and lock using the pivoting arrangement 111 to correlate or match with the position of the sun or the user's preference. The pivoting arrangement 111 enables the mount 110 to selectively position the filters relative to the user. The user may also use the pivoting arrangement 111 to obtain a preferred height and angle difference between themself and the filters to adapt to (or for) a variety of factors. By implementing these aspects of the disclosure, the tanning apparatus 100 may accommodate a variety of body types, sun angles and/or locations, seating and/or laying positions, etc.

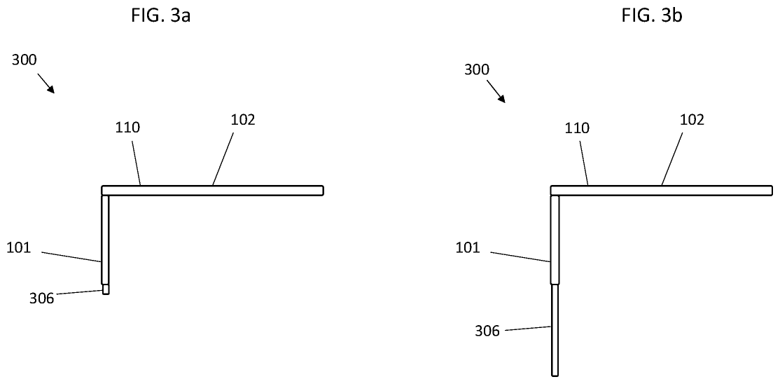

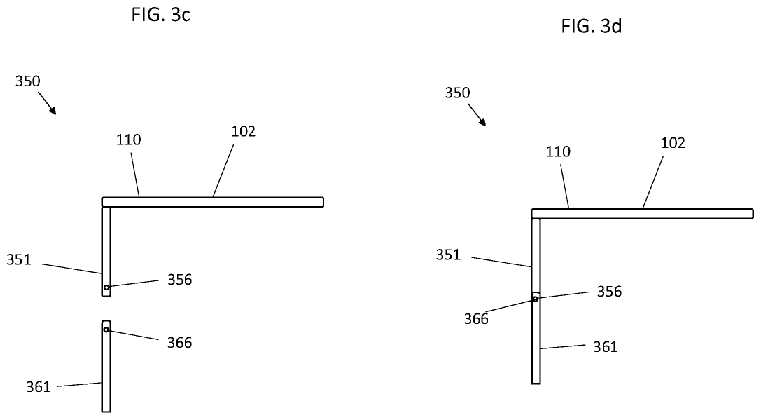

FIG. 3a shows a front view of an exemplary tanning apparatus 300 with the support 101 and a support extension 306 in an un-extended position in accordance with aspects of the disclosure. In embodiments, the support 101 may adjust (lengthen or shorten) in its axial direction. For example, the support 101 may telescope or expand in the axial direction by lengthening or extending the support extension 306. Additionally, the overall height of the tanning apparatus 300 may be adjusted or controlled based on an amount of axial extension of the support extension 306. In such a manner, for example, the height of the tanning apparatus may be adjusted to accommodate the desired tanning preferences of the user. For example, the support 101 may be adjusted based on the desired seating and/or lounging position of the user as well as the angle and/or position of the sun. By implementing aspects of the disclosure, the user may ensure that the tanning apparatus 300 is optimally positioned at an appropriate height and/or angle to achieve a desired sun and/or UV exposure. Additionally, the length of the support 101 may also be adjusted for ease of access into and out of cover from the tanning apparatus 300, storage, and/or any other desired reason for height adjustment of the tanning apparatus 300.

In exemplary embodiments, the extendable members (e.g., the support 101, the frame(s) 102, and/or the arm(s) 103, etc.) may include: telescopic members (e.g., extension members) and/or folding members. In accordance with aspects of the disclosure, in embodiments, the telescopic members may have an elongated rear outer tube that telescopically accepts a second front inner tube through a surface-to-surface frictional sliding movement, rolling movement, or other telescopic movement. In another exemplary embodiment, a front portion of the support 101, frame(s) 102, and/or arm(s) 103 may be hingedly attached to a rear portion for folding movement of the front portion relative to the rear portion about the hinge connection, as shown in FIG. 2a and FIG. 2b. In another exemplary embodiment, the support 101, the frame(s) 102, and/or the arm(s) 103 may comprise of two or more separate pieces attachable together. In accordance with aspects of the disclosure, in embodiments, each member (e.g., the support 101, the frame(s) 102, and/or the arm(s) 103, etc.) includes two or more separate pieces that may selectively join and/or connect (e.g., threadedly-attach, snap, fix, bolt, etc.). In embodiments, for example, the length of the mount 110 may be increased by joining two separate member pieces together, for example, by snap fitting together (or threadedly attaching) a male and female end of each of the member pieces.

FIG. 3b shows a front view of an exemplary tanning apparatus 300 with the support 101 and the support extension 306 in an extended position in accordance with aspects of the disclosure. In exemplary embodiments, the support extension 306 may extend or telescope out axially, from the support 101, to the extended configuration of FIG. 3b, from the un-extended configuration of FIG. 3a.

FIG. 3c shows a front view of an exemplary tanning apparatus 350 with a first support member 351 and a second support member 361 in an un-attached configuration in accordance with aspects of the disclosure. As shown in FIG. 3c, the tanning apparatus 350 may optionally include a first support member 351 with a female receiving end having an opening 356 and a second support member 361 having a male inserting end having a snap fitting 366. The first support member 351 and the second support member 361 may be selectively unattached, as shown in FIG. 3c, to limit the vertical height of the tanning apparatus 350 and/or facilitate transportation and/or storage.

FIG. 3d shows a front view of the exemplary tanning apparatus 350 with the first support member 351 and the second support member 361 in an attached configuration in accordance with aspects of the disclosure. As shown in FIG. 3d, the first support member 351 and the second support member 361 may be attached to one another by inserting the snap fitting 366 into the opening 356, to vertically increase the height of the tanning apparatus 350. In accordance with aspects of the disclosure, the first support member 351 and the second support member 361 may selectively join and/or connect by other methods (e.g., threaded connection, snap, fix, bolt, etc.). In accordance with aspects of the disclosure, other extendable members (e.g., frame(s), arm(s), etc.) may similarly connect to one or more additional corresponding members to increase in length.

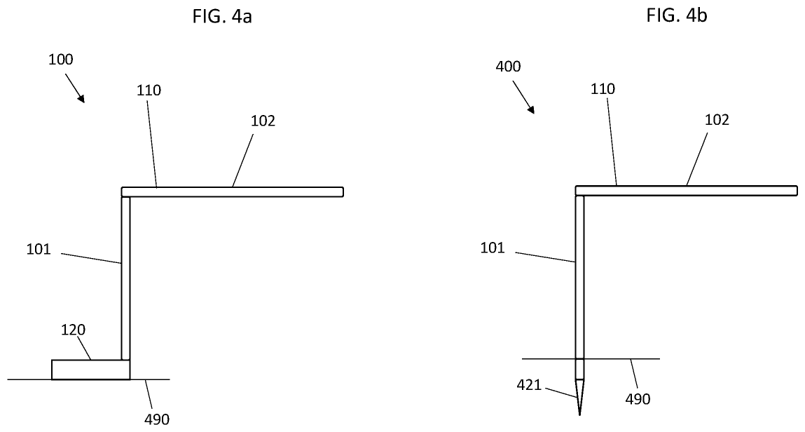

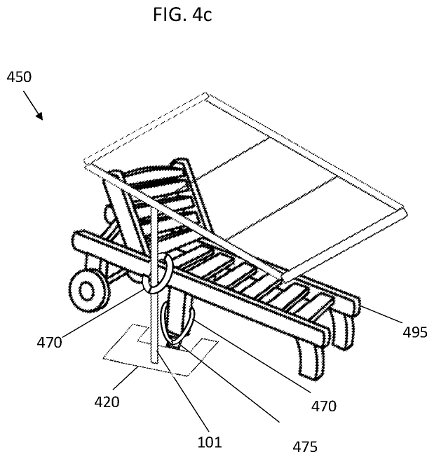

FIG. 4a shows a front view of an exemplary tanning apparatus 100 with a base 120 in accordance with aspects of the disclosure. As shown in FIG. 4a, the tanning apparatus 100 may optionally include a base 120 for holding the support 101 and mount 110 upright on a ground surface 490. The base 120 may be configured to sit atop the ground 490 and may be adapted to hold the support 101, including the mount 110, and any number of desired filters in an upright position. The base 120 may have sufficient mass and volume to counterbalance the weight and downward pull of the support 101, the mount 110, and the filter(s). FIG. 4a shows the base 120 attached to the bottom of the support 101 and extending approximately perpendicularly from the support 101 in a direction opposite of the mount 110. This configuration maximizes space for a user under the mount 110. The location of the base 120, however, may not be limited to one position with respect to the support 101 and may be configured or moved to any position or arrangement along the bottom of the support 101.

FIG. 4b shows a front view of another exemplary embodiment of a tanning apparatus 400 with a stake 421 in accordance with aspects of the disclosure. As shown in FIG. 4b, the bottom of the support 101 may be configured as a pointed stake 421 for penetrating into the ground 490 (e.g., sand). In this regard, the support 101 can be attached, fastened, or formed with a substantially pointed stake 421, adapted to be driven or otherwise received within the ground 490 (e.g., sand). The stake 421 may be configured so that the tanning apparatus 400 may be supported directly within the ground 490 (e.g., sand). The stake 421 is of sufficient length and/or surface area to secure the support 101 to the ground 490 (e.g., sand) while maintaining the support 101, and thus the mount 110, in any desired upright position. In embodiments, the stake 421 may be selected from, for example, a corkscrew stake, straight stake, etc.

FIG. 4c shows a perspective view of an exemplary tanning apparatus 450 with support fasteners 470 which may be attached to the base 420 and/or the support 101 in accordance with aspects of the disclosure. As shown in FIG. 4c, the base 420 includes a loop opening 475 for attaching a support fastener 470 that can be clipped to, looped around, and/or otherwise attached to separate objects/items (e.g., umbrella, chair, table, etc.) for securing thereto and/or support therefrom. Additionally, as shown in FIG. 4C, a second support fastener 470 can be clipped to, looped around, and/or otherwise attached to the support 101 and separate objects/items (e.g., umbrella, chair, table, etc.) for securing thereto and/or support therefrom. For example, as shown in FIG. 4C, the support fasteners 470 may loop around an adjacent lounge chair 495 to provide additional support. The support fastener 470 may include clips, snaps, rope, cord, cables, string, thread, tape, and/or hook and loop fasteners, etc., for releasably securing the base 420 and/or support 101 to a separate object.

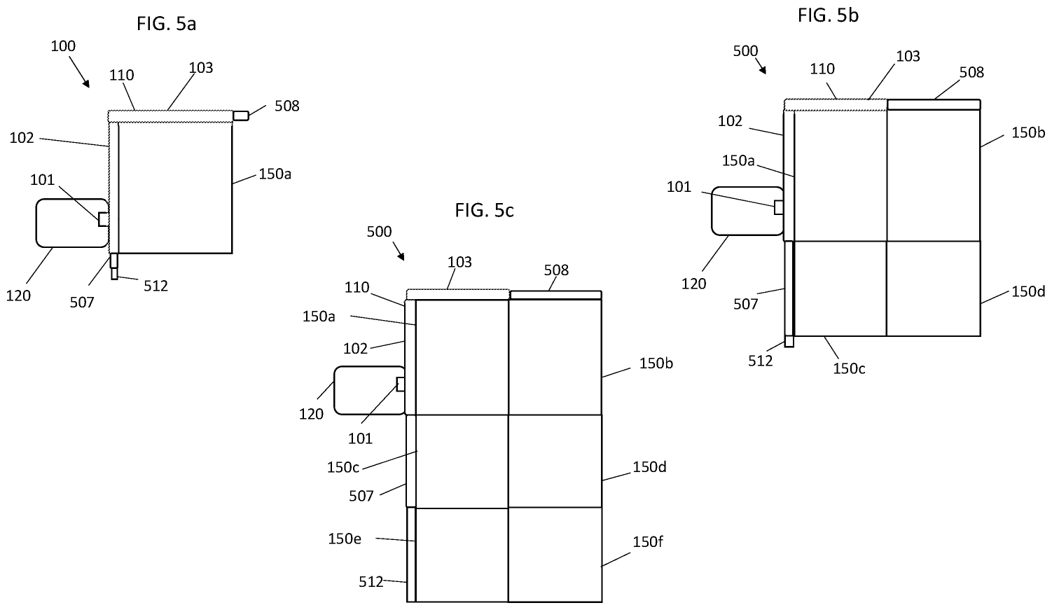

FIG. 5a shows a top view of an exemplary tanning apparatus 500 with a single filter 150a in accordance with aspects of the disclosure. As shown in FIG. 5a, the frame 102 may have a first frame extension 507 and a second frame extension 512 both configured in un-extended positions. Additionally, as shown in FIG. 5a, the arm 103 may have an arm extension 508 configured in an un-extended position. In this configuration the mount 110 may be configured to accommodate or hold a single filter 150a. With the exemplary embodiment of FIG. 5a, the first frame extension 507 and the arm extension 508, respectively, may be configured to selectively extend and retract in respective axial directions.

FIG. 5b shows a top view of an exemplary tanning apparatus 500 with four filters, 150a, 150b, 150c, and 150d, in accordance with aspects of the disclosure. As shown in FIG. 5b, the first frame extension 507 and the arm extension 508 may be configured in an extended position to accommodate attachment of additional filters 150, as compared to the un-extended position of FIG. 5a. As shown in FIG. 5b, filters 150a, 150b, 150c, and 150d attach to the mount 110, including to the first frame extension 507 and the arm extension 508. With the exemplary embodiment of FIG. 5b, the second frame extension 512 may be configured to selectively extend and retract in respective axial directions.

FIG. 5c shows a top view of an exemplary tanning apparatus 500 with six filters, 150a, 150b, 150c, 150d, 150e, 150f, in accordance with aspects of the disclosure. As shown in FIG. 5c, the second frame extension 512 is configured in an extended position from the extended position of the first frame extension 507 to accommodate attachment of additional filters 150. For example, FIG. 5c, shows filters 150a, 150b, 150c, 150d, 150e, and 150f attached to the mount 110, including to the second frame extension 512.

The exemplary embodiments of FIGS. 5a-5c show various configurations in which the frame 102 (including the first frame extension 507 and the second frame extension 512) and the arm 103 (including the arm extension 508) can be adjusted, retracted, or extended to accommodate different sizes and/or additional filters 150. Additionally, FIGS. 5b-5c show that some filters are not supported by either frames or arms but by other filter(s) 150. FIG. 5b shows, for example, the filter 150d attached to and supported by the filter 150b and the filter 150c while not in direct connection with either of the frame 102 or the arm 103. Thus, some of the filters 150 may not require attachment to the mount 110.

In an exemplary embodiment, the frame(s) 102 and/or arm(s) 103 may be adjusted or expanded to account for one or more of a variety factors. For example, the frame(s) 102 and/or the arm(s) 103 may be adjusted or extended to accommodate the attachment of additional filters, as shown in FIGS. 5b-5c, to customize tanning and/or the amount of sun/UV exposure to select areas or regions. Additionally, the frame(s) 102 and/or the arm(s) 103 may be adjusted or extended depending on the position or angle of the sun during tanning to keep consistent coverage during a tanning session. Further, the frame(s) 102 and/or the arm(s) 103 may be adjusted or extended to accommodate the length, width or height of the user, and further help maintain uniform and consistent coverage for a variety of different users. The frame(s) 102 and/or the arm(s) 103 may also be adjusted or extended for any other desired reason or motivation of the user.

In an exemplary embodiment, the support 101 may be a single supporting pole or post that extends up from the ground and supports the mount 110, as shown in FIG. 1. The single support structure enables the user 140 to more easily maneuver beneath and out of cover of the tanning apparatus 100. In accordance with aspects of the disclosure, with the single support structure, the tanning apparatus 100 is more portable and easily stored when not in use.

In an exemplary embodiment, various filters 150 may be attachable and/or removable from the mount 110. In exemplary embodiments, filters 150 that are suitably sized, may be placed on or attached to the frame via a fastener 870 (e.g., clips, snaps, hook and loop fasteners, and/or magnets). In additional embodiments, the mount 110 includes channels 890/891 or openings into which the filters 150 may selectively be inserted or slid into. By increasing the size of the mount 110 by expanding the frame(s) 102 and/or arm(s) 103, the mount 110 may be configured to support additional filters 150 and/or larger filters.

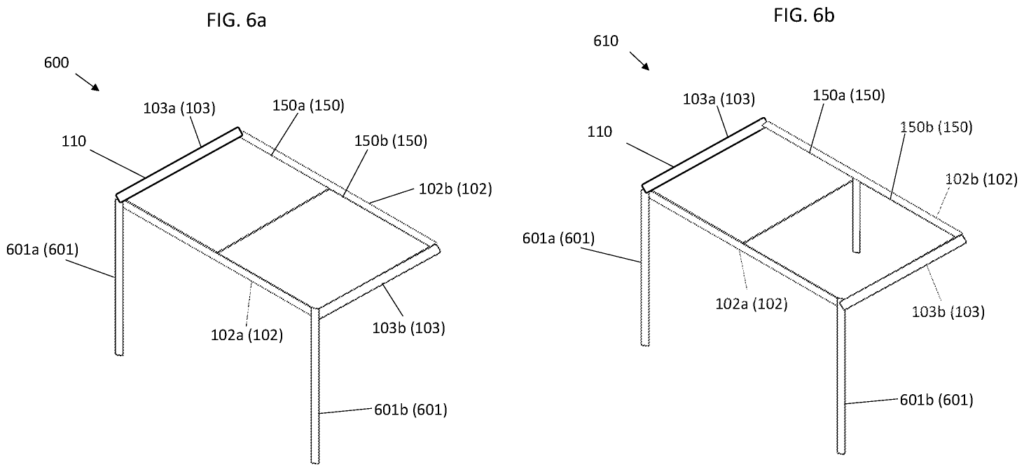

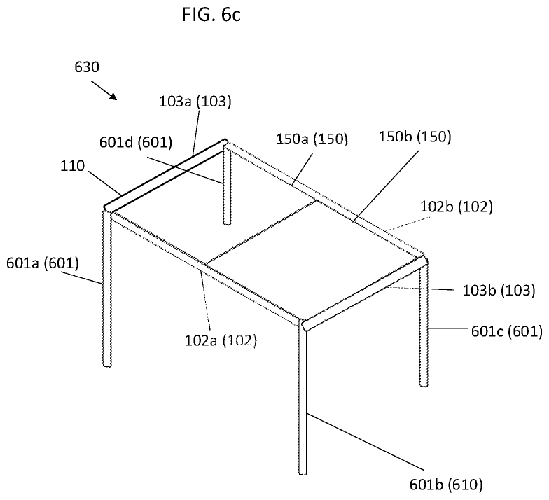

FIG. 6a shows a top view of an exemplary tanning apparatus 600 with two supports 601 in accordance with aspects of the disclosure. As shown in FIG. 6a, the tanning apparatus 600 may be configured to have a first support 601a connecting to one end of the first frame 102a and a second support 601b connecting to an opposing end of the first frame 102a. The first support 601a and the second support 601b provide additional stability and support of the mount 110 (e.g., during windy conditions and/or when more (or heavier) filters 150 are attached to the mount 110).

FIG. 6b shows a top view of an exemplary tanning apparatus 610 with three supports 601 in accordance with aspects of the disclosure. As shown in FIG. 6b, the tanning apparatus 610 is configured with a first support 601a connecting to one end of the first frame 102a and a second support 601b connecting to an opposing end of the first frame 102a. Additionally, the tanning apparatus 610 may be configured with a third support 601c connecting to the second frame 102b (e.g., approximately center position of the second frame 102b). The first support 601a, the second support 601b, and the third support 601c enable more stability and support for the mount 110 than the embodiment of FIG. 6a but has the added weight of an additional support 601c.

FIG. 6c shows a top view of an exemplary tanning apparatus 630 with four supports 601 in accordance with aspects of the disclosure. As shown in FIG. 6c, the tanning apparatus 630 is configured with a first support 601a connecting to one end of the first frame 102a and a second support 601b connecting to an opposing end of the first frame 102a. Additionally, the tanning apparatus 630 may be configured with a third support 601c and a fourth support 601d, each respectively connecting to opposing ends of the second frame 102b. The first support 601a, the second support 601b, the third support 601c, and the fourth support 601d provide even more stability and support for the mount 110 than either of the embodiments of FIG. 6a and FIG. 6b (but may weigh more).

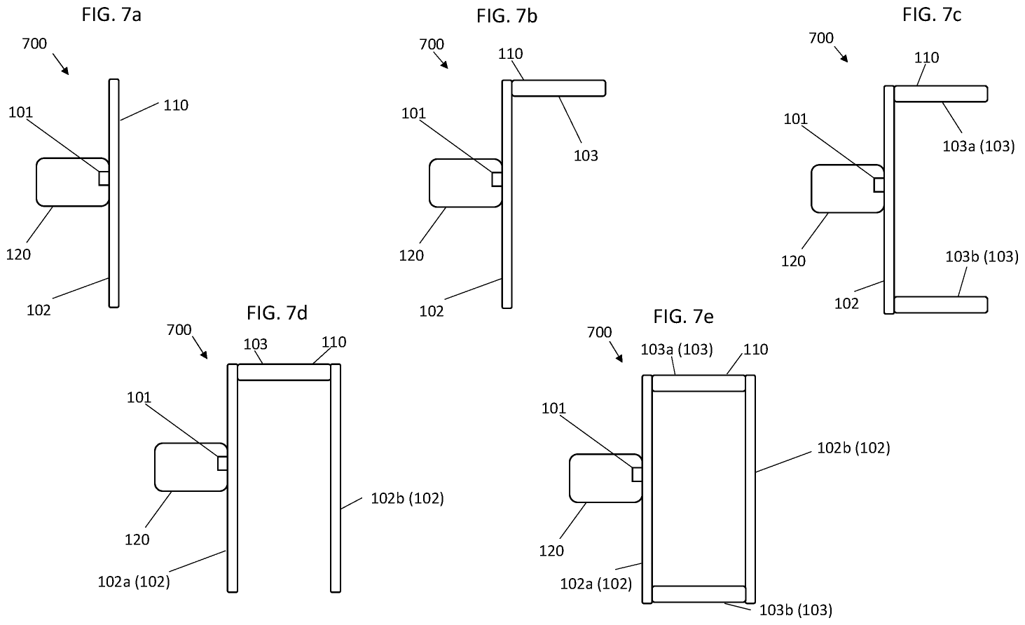

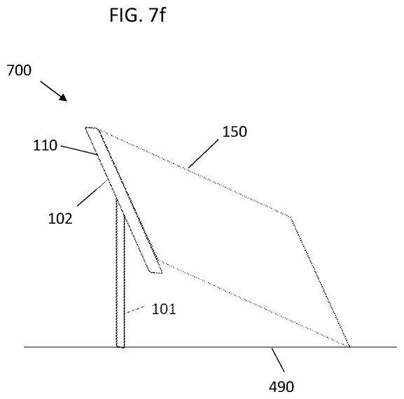

FIGS. 7a-7f show a single embodiment of an exemplary tanning apparatus 700 configured in any of 5 configurations depending on current need.

FIG. 7a shows a top view of the exemplary tanning apparatus 700 with a mount 110 having a single frame 102 in accordance with aspects of the disclosure. As shown in FIG. 7a, the mount 110 includes a single frame 102. The one or more filters may be rigid and/or planar and are configured to attach to the frame 102. In an embodiment, the filter(s) may be planar or flat when supported by the mount 110 such that the filter(s) occupy a single plane across the mount 110. The filter(s) may also be rigid so that the filter(s) are self-supporting and require less external support from the mount 110. In embodiments having a rigid filter, the filter(s) may require support from only a single side. The tanning apparatus 100 with the single frame 102 configuration shown in FIG. 7a, may be lighter in weight than the other exemplary embodiments and thus may be easier to transport and set-up as a portable embodiment.

FIG. 7b shows a top view of an exemplary tanning apparatus 700 with a mount 110 in an L-shape configuration in accordance with aspects of the disclosure. As shown in FIG. 7b, the mount 110 may be a single frame 102 connected to a single arm 103 at one end of the frame 102, to form an approximate L-shape. In an embodiment, the one or more filters may be configured to attach and be supported approximately parallel to the ground on the frame 102 and arm 103. This embodiment provides the benefit of additional support from the structure of FIG. 7a (however it may weigh more).

FIG. 7c shows a top view of an exemplary tanning apparatus 700 with a mount 110 in a C-shape configuration in accordance with aspects of the disclosure. As shown in FIG. 7c, the mount 110 may be a single frame 102 connected to a first arm 103a, at one end of the frame 102, and a second arm 103b, at the opposing end of the frame 102, to form an approximate C-shape. In an embodiment, the one or more filters may be configured to attach and be supported approximately parallel to the ground on the frame 102 and between the first arm 103a and the second arm 103b. This embodiment provides the benefit of additional support from the structures of FIG. 7a and FIG. 7b (however it may weigh more).

FIG. 7d shows a top view of an exemplary tanning apparatus 700 with a mount 110 in a U-shape configuration in accordance with aspects of the disclosure. As shown in FIG. 7d, the mount 110 may be a first frame 102a and a second frame 102b connected to each other by a single arm 103 at respective ends to form an approximate U-shape (or upside-down U-shape). In an embodiment, the one or more filters may be configured to attach and be supported approximately parallel to the ground on the arm 103 and between the first frame 102a and the second frame 102b.

FIG. 7e shows a top view of an exemplary tanning apparatus 700 with a mount 110 in a rectangular configuration in accordance with aspects of the disclosure. As shown in FIG. 7e, the mount 110 may be a first frame 102a and a second frame 102b connected to each other by a first arm 103a and a second arm 103b. In accordance with aspects of the disclosure, the first arm 103a and the second arm 103b may attach at respective opposing ends of the frames 102, to form a substantially rectangular shape, as shown in FIG. 7e. This embodiment provides the most amount of support (e.g., for supporting the filter(s)) (but may have increased weight).

FIG. 7f shows a perspective view of an exemplary tanning apparatus 700 with a tent-like configuration in accordance with aspects of the disclosure. In an embodiment, as shown in FIG. 7f, the mount 110 includes a support 101, a single frame 102, and a filter 150. The filter 150 may be supported by the frame 102 and the ground 490, to form a tent-like configuration, as shown in FIG. 7f. This embodiment provides a lightweight configuration.

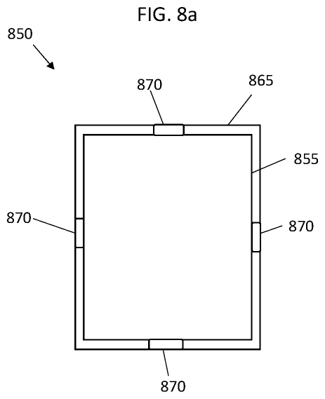

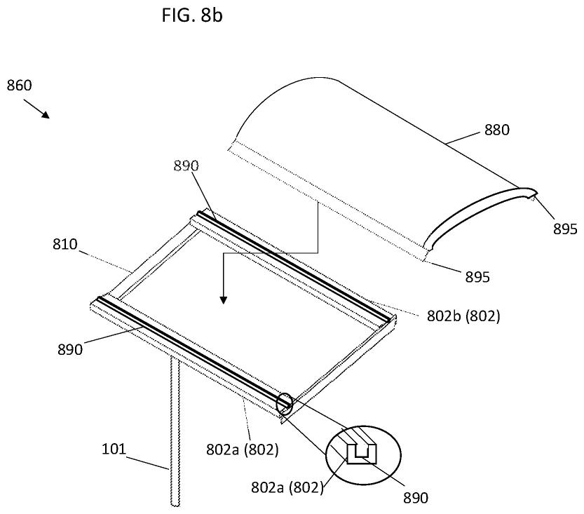

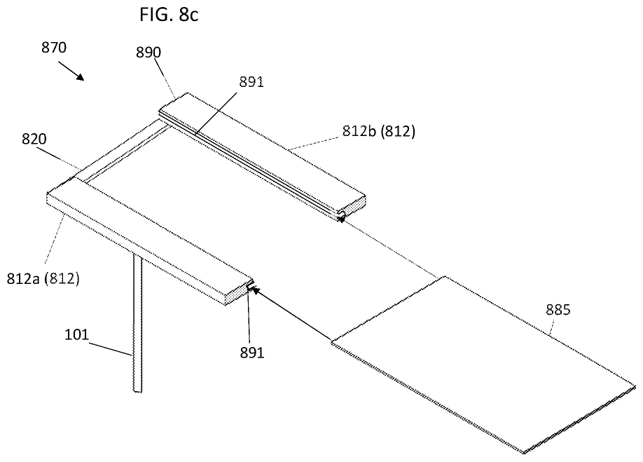

FIG. 8a shows a top view of an exemplary filter 850 with a fixture 865 in accordance with aspects of the disclosure. In exemplary embodiments, as shown in FIG. 8a, the filter 850 includes a fixture 865 that surrounds a filter material 855 and includes one or more fasteners 870. The fixture 865 may provide additional support, for example, when the filter material 855 is made from light weight, flexible, or pliable material. The fixture 865 may be rigid and/or semi-rigid light weight material that may provide additional support to the filter material 855 with minimal weight. The fixture 865 may be, for example, metal, plastic, rubber, and/or webbing material, etc., to provide support for the filter 850 along the perimeter (or portions) thereof. Additionally, the fixture 865 may include one or more fasteners 870 to connect or adjoin the filter 850 to one or more of the frame(s), arm(s), and/or additional filter(s) 850. The fasteners 870 can include clips, magnets, snaps, and/or hook and loop fasteners, etc., for releasably securing the filter(s) 850 to the mount and/or other filters 850.

As shown in FIGS. 1, 5a-5c, 6a-6c, and 9, the mount 110 may be configured to removably hold a single filter 150 or multiple filters 150a, 150b, 150c, etc. The one or more filters 150 may be UV filtering materials. The filters 150 may block, filter, or otherwise modify a percentage or amount of sunlight, specific wavelengths of light, and/or UV penetration. In embodiments, the filters 150 are configured to be supported on the mount 110 and selectively filter an amount or percentage of UV from sunlight passing through the filter 150 to the user 140 sun tanning below. The filter(s) 150 may be attachable to the mount 110 so that they can be easily interchanged and/or adjusted on the mount 110. The filter(s) 150 may be attached to the mount 110 by a fastener 870. The fastener 870 can include clips, magnets, snaps, and/or hook and loop fasteners, etc., for releasably securing the filter(s) 150 to the mount 110 and/or another filter 150. In embodiments, the fastener(s) 870 of the filter(s) 150 may selectively attach to a corresponding magnetic fastener 870 or ferromagnetic region (not shown) of the mount 110 to secure the filter(s) 150. In another embodiment, the fastener(s) 870 are hook and loop fasteners and the fastener(s) 870 of the filter(s) 150 may selectively wrap around a portion of the mount 110 to connect to itself.

FIG. 8b shows a perspective view of an exemplary mount 810 with channels 890 for attaching an exemplary filter 880 with ridges 895 in accordance with aspects of the disclosure. In exemplary embodiments, as shown in FIG. 8b, each frame 802 of the mount 810 may have at least one slot or channel 890 along the surface that corresponds to ridges 895 of a filter 880. With this exemplary embodiment, the ridges 895 of the filter 880 are approximately perpendicular to the face of the filter 880 and are located along at least portions of the perimeter. In this embodiment, one or more filters 880 may slide into or be inserted into the channels 890 of the mount 810 for securing the filter(s) 880. In embodiments, any number of channels 890 may be located along any part or portion of the mount 810 (e.g., frames, arms) to correspond with the location and size of the ridges 895 of the filter(s) 880. This embodiment may secure the filters 880 to the mount 810 without additional fasteners or securing elements.

FIG. 8c shows a perspective view of an exemplary mount 820 with slide channels 891 for sliding in an exemplary filter 885 in accordance with aspects of the disclosure. In exemplary embodiments, as shown in FIG. 8c, at least one frame 812 of the mount 820 may have at least one slot or slide channel 891 along the filter facing inwardly arranged surface. In this embodiment, the thickness of the slide channel may correspond in thickness to the filter 885, so that the filter 885 may slide in and be secured within the slide channel 891. In embodiments, any number of slide channels 891 may be located along any part or portion of the mount 820 (e.g., frames, arms) to correspond with the location and size of the filter(s) 885. This embodiment may secure the filters 885 to the mount 820 without additional fasteners or securing elements.

In an exemplary embodiment, the mount 110 may be configured so that any variety or number of filters 150 may be attached to the mount 110 at one time. By enabling multiple different filters 150 to attach to the mount 110, the user 140 may control a variety of tanning zones to selectively control the amount of light exposure or level of tanning for different portions or areas of their body.

In exemplary embodiments, the filters 150 may be made of any suitable material capable of light wavelength or UV filtering, including but not limited to glass, plastics, acrylics, and films. The filters 150 may be rigid and planar structures such as sheets of glass or hard acrylic. In alternative embodiments, the filters 150 may be pliable and/or flexible (e.g., roll of film). In embodiments, the filters 150 may be convex or concave such that the filter material is bent or curved to provide additional support and/or alter or otherwise modify the sunlight and/or UV penetration directed to a user.

In exemplary embodiments, the filters 150 may be clear or varied tints and/or colors to affect UV and/or visible light penetration. In accordance with aspects of the disclosure the filter 150 may be photochromatic, electrochromatic (smart film), or may be a non-changing material.

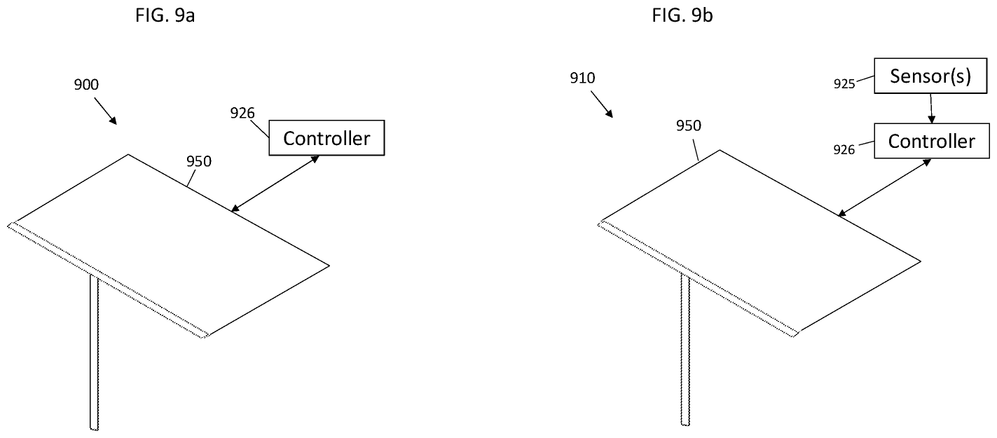

FIG. 9a shows an exemplary tanning apparatus 900 with a user-selectable film tinting and/or smart-film filter 950 in accordance with aspects of the disclosure. In embodiments, as shown in FIG. 9a, in embodiments the tanning apparatus 900 may include a controller 926 and a smart-film filter 950. The controller 926 is operable to selectively adjust the tinting and/or UV/light filtering of the smart-film filter 950. The controller 926 may be a wireless or wired controller (e.g., mobile phone or a manual switch). The control signal for adjusting the tinting and/or UV/light filtering may be based on instructions from a user (e.g., via manual switch, mobile phone, voice control). For example, the tinting and/or UV/light filtering of the smart-film filter 950 may be adjusted by the controller 926 to match the tanning parameters of a user. In exemplary embodiments, the smart-film filter 950 may have an auto shut-off feature. In this embodiment, the controller would enable the smart-film feature to become opaque and/or block the sun after a period of time, in correlation with a timed end of a tanning session. This feature would help prevent sun burn and/or excessive sun exposure.

FIG. 9b shows an exemplary tanning apparatus 910 with a smart-film filter 950 in accordance with aspects of the disclosure. In embodiments, as shown in FIG. 9b, the tanning apparatus 910 may include one or more sensor(s) 925, a controller 926, and a smart-film filter 950. The controller 926 is operable to automatically adjust the tinting and/or UV/light filtering of the smart-film filter 950 based on signals received by the sensor 925. In embodiments, the tinting and/or UV/light filtering may be adjusted based on signal(s) received by the sensor(s) 925, which may indicate, for example, the position and/or angle of the sun, UV index, and/or surrounding reflections, etc. This embodiment helps ensure consistent coverage and sun exposure over varying conditions.

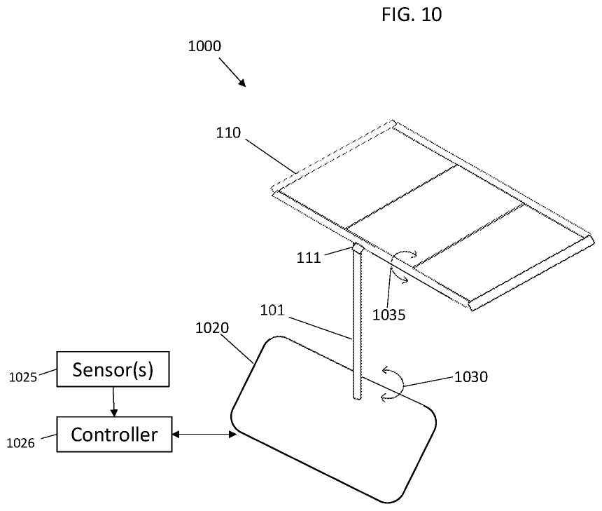

FIG. 10 shows an exemplary tanning apparatus 1000 with a smart base 1020 in accordance with aspects of the disclosure. As shown in FIG. 10, the tanning apparatus 1000 may include a smart base 1020 that is connected to one or more sensor(s) 1025 and a controller 1026. In accordance with aspects of the disclosure, the controller 1026 may cause selective operation of the smart base 1020 to actuate motion and/or operation of the support 101. In additional embodiments, the tanning apparatus 1000 may have selective rotation between the support 101 and the mount 110. In embodiments, operation of the smart base 1020 may cause selective motion and/or rotation of the support 101 and corresponding mount 110. In further embodiments, actuation of the controller 1026 may selectively control rotation between the support 101 and the mount 110. As shown in FIG. 10, the mount 110 is connected to the support 101 by the pivoting arrangement 111 for adjustably pivoting and locking the mount 110 at a plurality of positions and/or angles (e.g., tilt adjustment in direction 1035). In exemplary embodiments, the smart base 1020 may cause selective tilt adjustment of the mount 110 in a direction 1035 to provide more consistent coverage from the sun. In embodiments, the smart base 1020 can receive input from the controller 1026 to actuate the support 101 to rotate (in direction 1030) and align with the angle of the sun. In additional embodiments, the support 101 may be configured (e.g., with a servo motor) to actuate the mount 110 to rotate (in direction 1030) and align with the angle of the sun. The controller 1026 may correspond to a mobile phone or application. The smart base 1020 may automatically adjust the support 101 and/or the mount 110 (by the pivoting arrangement 111), to changing positions of the sun based on signals received from the sensor(s) 1025. In embodiments, the controller 1026 may use signals, for example, associated with light angle and/or intensity, as received by the sensor(s) 1025, to control the rotational position of the support 101 and/or mount 110, as well as the angular (tilt) position of the mount 110. In accordance with aspects of the disclosure, this configuration enables consistent coverage and tanning for the user.

In exemplary embodiments, the controller 926/1026 may be programmed to use the sensor(s) 925/1025 signals to adjust one or both of the smart film filter 950 and/or the smart base 1020. In additional embodiments, the controller 926/1026 may use voice control to adjust one or both of the smart film filter 950 and/or the smart base 1020 by voice command of the user. In another exemplary embodiment, the controller 926/1026 may use a timer to adjust one or both of the smart film filter 950 and/or the smart base 1020 so that the support 101 and corresponding mount 110 adjusts over specific time intervals.

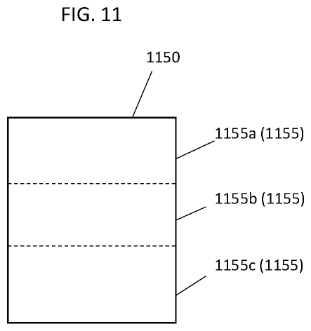

FIG. 11 shows a top view of an exemplary filter 1150 with multiple filter regions 1155 in accordance with aspects of the disclosure. As shown in FIG. 11, the filter 1150 may include a first region 1155a, a second region 1155b, and a third region 1155c. Each region 1155 of the filter 1150 may have the same or different filtering properties such that each region 1155 may permit different amounts of tinting, wavelength blocking, UV/light filtering, and/or sunlight exposure. In accordance with aspects of the disclosure, for example, the first region 1155a may have a select filtering property for tanning the face of the user, the second region 1155b may have a different second selected filtering property for tanning the torso, and the third region 1155c may have a third different selected filtering property for tanning the lower extremities. The filter 1150 may include any variety, arrangement, or number of different regions 1155. In accordance with aspects of the disclosure, the filter regions 1155 may divide the filter 1150 from left to right and/or top to bottom.

In an exemplary embodiment the mount 110 may include a single filter 150 with uniform tinting or UV/light filtering. In another exemplary embodiment, shown in FIG. 1, the mount 110 may have a first filter 150a and a second filter 150b attached. Each of the filters 150 enable the same, varying, or different amounts of UV/light exposure so that, for example, respective upper and lower portions of the user may receive the same or different amounts of UV/sunlight exposure. In another exemplary embodiment, the mount 110 may have arranged thereon a first filter, a second filter, and a third filter serially arranged. This arrangement corresponds to at least three separate amounts of tinting or UV/light filtering, so that, by example, the face, torso, and leg regions of the user may experience different levels of UV or light exposure. Thus, the user may selectively control or designate the level of sun exposure for specific regions to effectively customize tanning. In an even further embodiment, as shown in FIG. 5b, the mount 110 may have a first filter 150a, a second filter 150b, a third filter 150c, and a fourth filter 150d attached. Each of the filters 150 may enable the same or different amounts of UV/light exposure so that four separate sections of the user (or users) may receive the same or different amounts of UV/sunlight exposure. Each of these embodiments is exemplary and non-limiting and any variety or number of different filters 150 may be attached to the mount 110 to selectively control the amount and variety of UV/sunlight exposure to various regions of the user.

In exemplary embodiments, the tanning apparatus 100 may be designed to be stand alone and/or portable. The support(s) 101, the mount 110, the frame(s) 102, the arm(s) 103, and the filter(s) 150 may be collapsible and expandable for ease of transportation to a tanning site (e.g., a beach). In other embodiments the tanning apparatus 100 may be more permanent for backyard pools and/or lounging areas.

In exemplary embodiments, the support(s) 101, the mount 110, the frame(s) 102, the arm(s) 103, and the filter(s) 150 may also be configured to be assemblable and/or made foldable in order to accommodate transportation of the tanning apparatus 100. Each filter 150 can be disconnected from the mount 110. In another exemplary embodiment, the support 101 may be pivotally connected to the mount 110, to allow the mount 110 to be folded inwards and/or expanded outwards from the support 101, to facilitate storage and transportation. The support(s) 101 may be connected to the mount 110, for example, by a locking hinge joint that allows the mount 110 to selectively fold down against the support(s) 101. In another embodiment, each of the support(s) 101, the frame(s) 102, and the arm(s) 103 may be telescoping in order to enable each component to be shortened to create a more compact unit for transportation and storage. The support(s) 101, the frame(s) 102, and the arm(s) 103 may also include a variety of locking features that allow each of the support(s) 101, the frame(s) 102, and the arm(s) 103 to be disconnected, pivoted, and/or collapsed for storage and transportation.

In exemplary embodiments, the filter(s) 150 may be interchangeable in various locations on the mount 110. According to aspects of the disclosure, each filter 150 location can be selected, for example, based on the user's desired degree of comfort, tanning speed, and/or desired UV protection on various parts, regions, and/or areas of the user. The filters 150 may be selected, for example, based on the specific UV absorption, sunlight reflection, transmission and/or shading properties desired to account for sun movement, sun intensity (e.g., based on geolocation) and environmental changes (e.g., clouds, wind, etc.) during the day. For example, the user may decide mid-way through a tanning session that more UV protection is desired and can interchange a more transparent or light transmissible filter for one that provides a greater amount of UV protection.

Additionally, in exemplary embodiments, each filter 150 may be selectively chosen to permit a specific percentage of light penetration. For example, one filter 150 may be chosen to have 5% light penetration percentage, whereas a separate filter 150 may have 50% light penetration. As shown in FIG. 1, each filter 150 may include an identifier or label 152 for identifying one or more properties (e.g., UV/sunlight blocking capability, percentage of light penetration, wind resistance strength) of the filters 150, and/or recommended times of skin exposure for the filters 150.

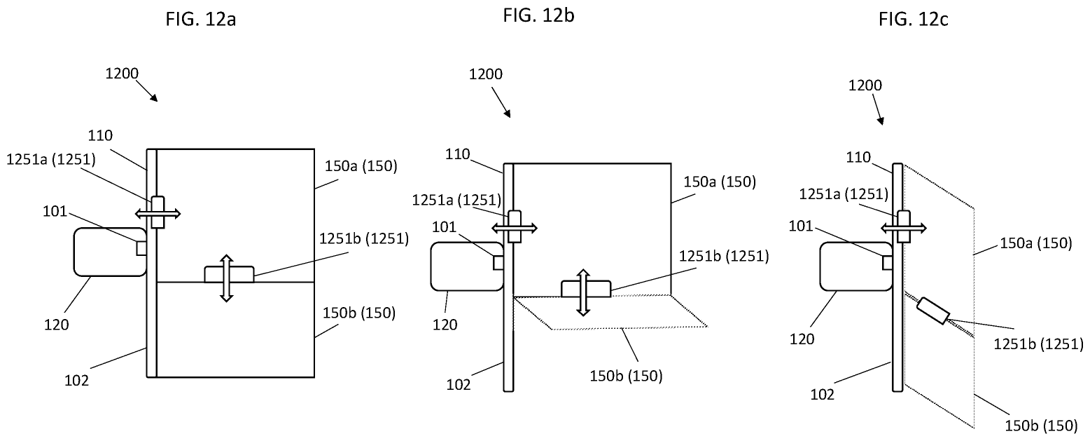

FIG. 12a shows a top view of an exemplary tanning apparatus 1200 with filters 150 connected with joints 1251 and positioned approximately perpendicular with respect to the mount 110 in accordance with aspects of the disclosure. As shown in FIG. 12a, the mount 110 and/or filters 150 may include one or more joints 1251 for selectively attaching each of the filters 150 to the mount 110 and/or other filters 150. Each joint 1251 may be fastened to both a filter 150 and a respective portion of the mount 110 or another filter 150, such that the joint 1251 is shared with both components. The joint 1251 may be, for example, a pivot, a hinge, a swivel joint, a locking joint, a ratcheting joint, a locking hinge, or a locking hinged joint to allow the filter 150 to selectively rotate, tilt, and/or pivot about the mount 110 and/or additional filter 150. The joint 1251 may be adapted for selective rotation of the filter 150 relative to the mount 110 and/or additional filter 150 for setting a position thereof. The joint 1251 may be selectively locked in any desired position. In an exemplary embodiment, FIG. 12a shows where a first joint 1251a is between a first filter 150a and the frame 102. The first filter 150a may be pivoted and/or rotated about the frame 102 along the rotational axis of the joint 1251a. FIG. 12a also shows a second joint 1251b attached to both the first filter 150a and the second filter 150b. In exemplary embodiments, each of the first filter 150a and the second filter 150b may be rotated and/or rotated on top of one another so that the first filter 150a and the second filter 150b may be in a stacked position. The joints 1251 can be in conjunction with or alternative to the pivoting arrangement.

FIG. 12b shows a top view of an exemplary tanning apparatus 1200 with the second filter 150b pivoted up approximately parallel with respect to the first filter 150a in accordance with aspects of the disclosure. In exemplary embodiments, the filters 150 may connect by a joint 1251, enabling the filters 150 to pivot and/or rotate about each other. In this configuration the position and/or angle of the second filter 150b may be configured to be selectively changed relative to the first filter 150a, as shown in FIG. 12b. This configuration also enables the filters 150 to be stackable so that the filtering properties may be combined and also enables easier storage and packing.

FIG. 12c shows a top view of an exemplary tanning apparatus 100 with a first filter 150a and a second filter 150b pivoted approximately at a 45-degree angle with respect to the frame 102 in accordance with aspects of the disclosure. In exemplary embodiments, the joint(s) 1251 enables the filter(s) 150 to pivot and/or rotate in relation to the mount 110. As shown in FIG. 12c, the first joint 1251a enables the first filter 150a and the second filter 150b to be pivoted to a different position from FIG. 12a. This configuration enables the user to easily change the position of one or more filters based on the position of the sun. Additionally, this configuration also enables the user to more easily maneuver beneath and out of cover of the filters 150, by rotating their position.

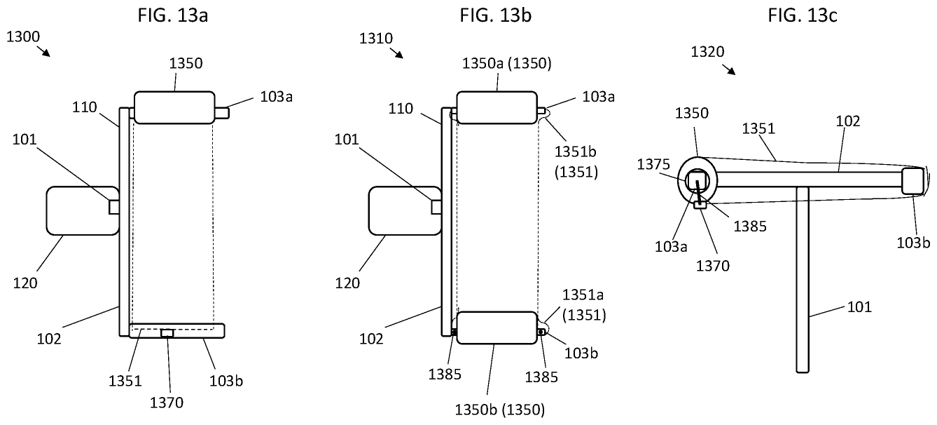

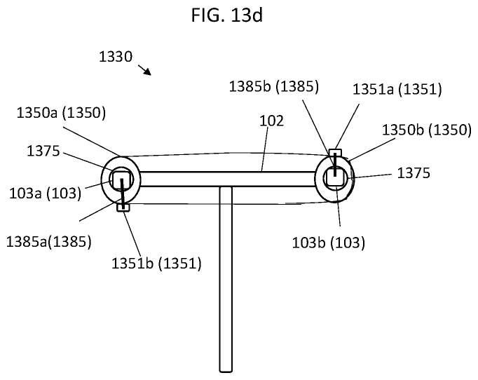

FIG. 13a shows a top view of an exemplary tanning apparatus 1300 with a roll filter 1350 in accordance with aspects of the disclosure. As shown in FIG. 13a, the apparatus 1300 may contain a roll filter 1350 so that the flexible and/or pliable filtering material may be configured to be unfurled along the length of the mount 110. The roll filter 1350 may be attached to the mount 110 by, for example, fasteners, clips, hook and loop fasteners, and/or protruding pins, etc. For example, as shown in FIGS. 13c-13d, the roll filters 1350 may be attached and supported by inserting the first arm 103a through a core support 1375 of the roll filter 1350. Additionally, an end portion 1351 of the roll filter 1350 may include at least one fastener 1370. The fastener 1370 may be configured to attach the end portion 1351 to a portion of the mount 110. For example, as shown in FIG. 13a, the end portion 1351 of the roll filter 1350 may be attached to the second arm 103b by the fastener 1370. In embodiments, the roll filter 1350 may be un-rolled (unfurled) from the first arm 103a and wrapped around the second arm 103b to secure the roll filter 1350 on opposing ends of the mount 110. The fastener 1370 may include clips, magnets, snaps, protruding pins, and/or hook and loop fasteners, etc., for releasably securing the roll filter 1350, including the end portion 1351 to the mount 110.

Additionally, in exemplary embodiments, the filter may be configured in a folded arrangement. In exemplary embodiments, the filter may be selectively attached and supported at one end of the mount 110 and then configured to be unfurled by unfolding (e.g., in a fan folded manner) the filter to the other end of the mount 110 to be selectively attached and supported by a fastener. The fastener may include clips, magnets, snaps, protruding pins, and/or hook and loop fasteners, etc., for releasably securing the filter to the mount 110.

FIG. 13b shows a top view of an exemplary tanning apparatus 1310 with a first roll filter 1350a and a second roll filter 1350b in accordance with aspects of the disclosure. As shown in FIG. 13b, each of the first roll filter 1350a and the second roll filter 1350b may be configured to be unfurled along the length of the mount 110 such that at least a portion of the first roll filter 1350a and the second roll filter 1350b overlap along the length of the mount 110. The end portions 1351 of each roll filter 1350 may be configured to attach to opposing ends of the mount 110. In exemplary embodiments, the first end portion 1351a of the first roll filter 1350a may be attached to an opposing end of the mount 110 by a pair of protruding pins 1385 that extend from each arm 103. The protruding pins 1385 are configured to hook through an opening of an elongated arm of the first end portion 1351a, as shown in FIG. 13b. Additionally, the second end portion 1351b of the second roll filter 1350b may be attached to the opposite end of the mount 110 by another respective pair of protruding pins 1385 that protrude in the opposite direction, as shown in FIG. 13d. The protruding pins hook through an opening of an elongated arm of the second end portion 1351b, as shown in FIG. 13b. This embodiment may provide two separate filter layers that overlap in order to further customize the filtering properties to the user's tanning preference.

FIG. 13c shows a front view of an exemplary tanning apparatus 1320 with a roll filter 1350 in accordance with aspects of the disclosure. As shown in FIG. 13c, the roll filters 1350 may be attached and supported to the tanning apparatus 1320 by inserting the first arm 103a through a core support 1375 of the roll filter 1350. Additionally, as shown in FIG. 13c, the end portion 1351 of the roll filter 1350 may be configured to wrap around the top and over the second arm 103b and attach to a pair of protruding pins 1385 that protrude from the first arm 103a and hook through an opening of an elongated arm of the end portion 1351. This embodiment may enable a reel-like configuration of the roll filter 1350 to provide a double layer of filtering material from a single roll filter 1350.

FIG. 13d shows a front view of an exemplary tanning apparatus 1330 with a first roll filter 1350a and a second roll filter 1350b in accordance with aspects of the disclosure. As shown in FIG. 13d, the first end portion 1351a is attachable to the second protruding pin 1385b of the second arm 103b. Additionally, as shown in FIG. 13d, the second end portion 1351b is attachable to the first protruding pin 1385a of the first arm 103a. This embodiment enables two separate overlapping filtering layers depending on the user's desired tanning preference.

In exemplary embodiments, the first roll filters 1350a and the second roll filter 1350b can be utilized separately or in tandem, such that the first roll filter 1350a and the second roll filter 1350b may not overlap, overlap in part, or overlap completely.

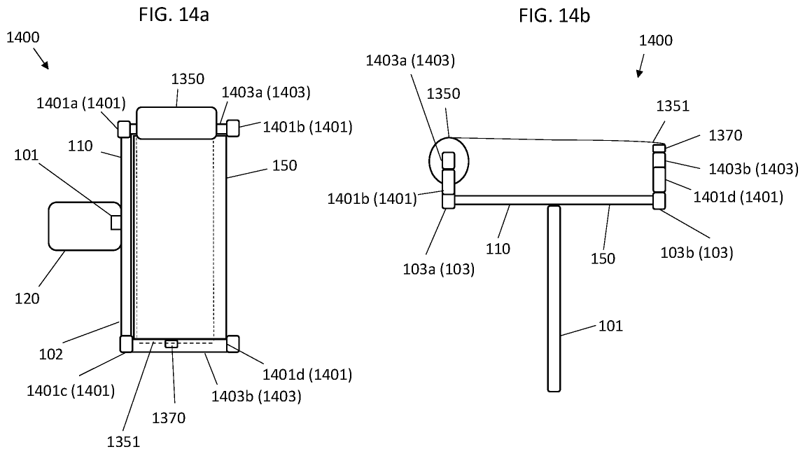

FIG. 14a shows a top view of an exemplary tanning apparatus 1400 with a roll filter 1350 and a planar filter 150 in accordance with aspects of the disclosure. As shown in FIG. 14a, the roll filter 1350 is configured to attach to a first elevated arm 1403a that is elevated by a first post 1401a and a second post 1401b. The end portion 1351 attaches to a second elevated arm 1403b by a fastener 1370. The second elevated arm is supported by a third post 1401c and a fourth post 1401d. The planar filter 150 may attach beneath the roll filter 1350 and is configured to be supported on the first arm 103a and the second arm 103b, as shown in FIG. 14b. This embodiment enables for further customization of tanning by being able to select and overlap both a planar filter 150 and a roll filter 1350.

FIG. 14b shows a front view of an exemplary tanning apparatus 1400 with a roll filter 1350 and a planar filter 150 in accordance with aspects of the disclosure. The roll filter 1350 may be elevated above the planar filter 150 by the first post 1401a and the second post 1401b that supports the first elevated arm 1403a. The first elevated arm 1403a is inserted through the middle opening of the roll filter 1350 to support and elevate the roll filter 1350 above the planar filter 150, as shown in FIG. 14b. The end portion 1351 is attached to the second elevated arm 1403b by the fastener 1370.

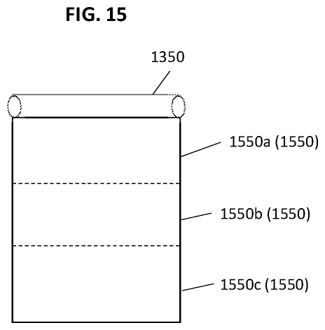

FIG. 15 shows a top view of an exemplary roll filter 1350 with multiple filter regions 1550 in accordance with aspects of the disclosure. As shown in FIG. 15, the roll filter 1350 may include a first region 1550a, a second region 1550b, and a third region 1550c. Each region 1550 of the roll filter 1350 may have the same or different filter properties, such that the different parts or regions of the filter may permit different amounts of tinting, wavelength blocking, UV/light filtering, and/or sunlight exposure. For example, the first region 1550a may have a select filtering property for tanning the face of the user, the second region 1550b may have a different second selected filtering property for tanning the torso, and the third region 1550c may have a third different selected filtering property for tanning the lower extremities of the user. The roll filter 1350 may include any variety, arrangement, or number of different regions 1550. The filter regions 1550 may divide the roll filter 1350 from left to right and/or top to bottom. For example, the first thirty-percent of the roll filter 1350 may have 15% light penetration percentage, the next thirty-percent of the roll filter 1350 may have 25% light penetration, and the following forty-percent may have 10% light penetration. The lengths and amounts of light penetration may be non-limiting, any variety and/or combination of area, size, and/or amounts of light penetration along the roll filter 1350 may be possible.

In exemplary embodiments, the mount 110 may additionally be configured to attach additional components thereto. For example, as shown in FIG. 1, a shade 180 may be attached to the mount 110 by a shade fastener 170 and may increase tanning/skin protection, shading, and/or privacy of the user 140. The shade 180 may be a curtain, blind, fabric, or generally opaque material that may at least partially obscure vision or light passing through the shade 180. The shade fastener 170 may be one or more of magnets, snaps, and/or hook and loop fasteners, etc., for releasably securing the shade 180 to the mount 110. The shade 180 may be secured to any portion and/or length of the mount 110. The shade 180 may be sized to entirely surround or only cover a portion of the mount 110.

Aspects of embodiments of the present disclosure (e.g., a tanning apparatus) can be implemented by such special purpose hardware-based systems that perform the specified functions or acts, or combinations of special purpose hardware and computer instructions and/or software, as described above. The control systems may be implemented and executed from either a server, in a client server relationship, or they may run on a user workstation with operative information conveyed to the user workstation. In an embodiment, the software elements include firmware, resident software, microcode, etc. In contemplated embodiments, the control systems may be embedded in the tanning apparatus to make it stand alone. In contemplated embodiments, the tanning apparatus and control system may be virtually implemented on a touch screen.

As will be appreciated by one skilled in the art, aspects of the present disclosure may be embodied as a system, a method or a computer program product. Accordingly, aspects of embodiments of the present disclosure may take the form of an entirely hardware embodiment, an entirely software embodiment (including firmware, resident software, micro-code, touch screen, etc.) or an embodiment combining software and hardware aspects that may all generally be referred to herein as a “circuit,” “module” or “system.” Furthermore, aspects of the present disclosure (e.g., control systems) may take the form of a computer program product embodied in any tangible medium of expression having computer-usable program code embodied in the medium.

Any combination of one or more computer usable or computer readable medium(s) may be utilized. The computer-usable or computer-readable medium may be, for example but not limited to, an electronic, magnetic, optical, electromagnetic, infrared, touch screen, or semiconductor system, apparatus, device, or propagation medium. More specific examples (a non-exhaustive list) of the computer-readable medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CDROM), an optical storage device, a transmission media such as those supporting the Internet or an intranet, a magnetic storage device, a USB key, Bluetooth, and/or a mobile phone.

In the context of this document, a computer-usable or computer-readable medium may be any medium that can contain, store, communicate, propagate, or transport the program for use by or in connection with the instruction execution system, apparatus, or device. The computer-usable medium may include a propagated data signal with the computer-usable program code embodied therewith, either in baseband or as part of a carrier wave. The computer usable program code may be transmitted using any appropriate medium, including but not limited to wireless, wireline, optical fiber cable, RF, etc.

Computer program code for carrying out operations of the present disclosure may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer, entirely embedded within the tanning apparatus, or entirely on the remote computer or server. In the latter scenario, the remote computer may be connected to the user's computer through any type of network. This may include, for example, a local area network (LAN) or a wide area network (WAN), or the connection may be made to an external computer (for example, through the Internet using an Internet Service Provider). Additionally, in embodiments, the present disclosure may be embodied in a field programmable gate array (FPGA).

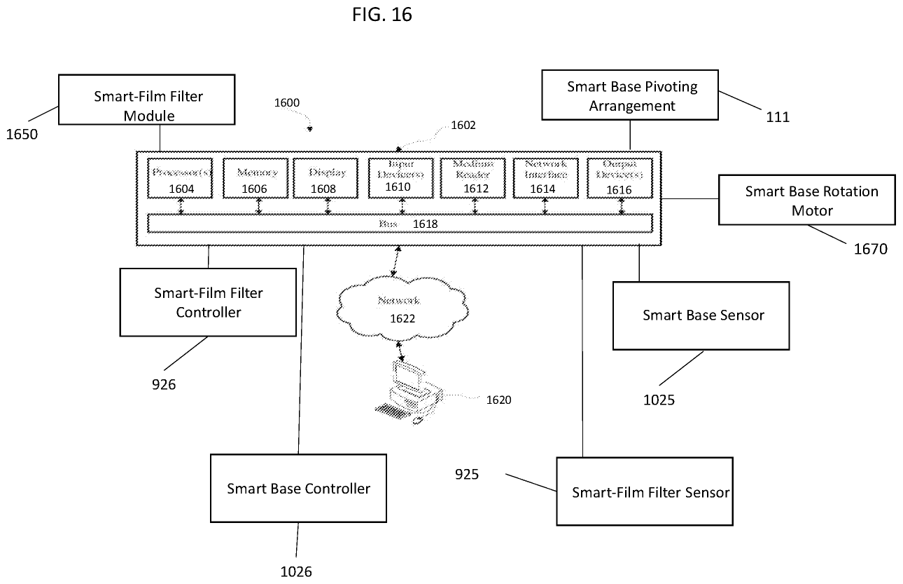

FIG. 16 is an exemplary system for use in accordance with the embodiments described herein. The system 1600 is generally shown and may include a computer system 1602, which is generally indicated. The computer system 1602 may operate as a standalone device or may be connected to other systems or peripheral devices. For example, the computer system 1602 may include, or be included within, any one or more computers, servers, systems, communication networks, cloud environment or embedded within the tanning apparatus.

The computer system 1602 may operate in the capacity of a server in a network environment, or in the capacity of a client user computer in the network environment. The computer system 1602, or portions thereof, may be implemented as, or incorporated into, various devices, such as a personal computer, a tablet computer, a set-top box, a personal digital assistant, a mobile device, a palmtop computer, a laptop computer, a desktop computer, a communications device, a wireless telephone, a personal trusted device, a web appliance, or any other machine capable of executing a set of instructions (sequential or otherwise) that specify actions to be taken by that device. Further, while a single computer system 1602 is illustrated, additional embodiments may include any collection of systems or sub-systems that individually or jointly execute instructions or perform functions.

FIG. 16 shows an exemplary environment for practicing aspects of the present disclosure. As illustrated in FIG. 16, the computer system 1602 may include at least one processor 1604, such as, for example, a central processing unit, a graphics processing unit, or both. The computer system 1602 may also include a computer memory 1606. The computer memory 1606 may include a static memory, a dynamic memory, or both. The computer memory 1606 may additionally or alternatively include a hard disk, random access memory, a cache, or any combination thereof. Of course, those skilled in the art appreciate that the computer memory 1606 may comprise any combination of known memories or a single storage.

As shown in FIG. 16, the computer system 1602 may include a computer display 1608, such as a liquid crystal display, an organic light emitting diode, a flat panel display, a solid state display, a cathode ray tube, a plasma display, or any other known display. The computer system 1602 may include at least one computer input device 1610, such as a keyboard, a remote control device having a wireless keypad, a microphone coupled to a speech recognition engine, a camera such as a video camera or still camera, a cursor control device, or any combination thereof. Those skilled in the art appreciate that various embodiments of the computer system 1602 may include multiple input devices 1610. Moreover, those skilled in the art further appreciate that the above-listed, exemplary input devices 1610 are not meant to be exhaustive and that the computer system 1602 may include any additional, or alternative, input devices 1610.

The computer system 1602 may also include a medium reader 1612 and a network interface 1614. Furthermore, the computer system 1602 may include any additional devices, components, parts, peripherals, hardware, software or any combination thereof which are commonly known and understood as being included with or within a computer system, such as, but not limited to, an output device 1616. The output device 1616 may be, but is not limited to, a speaker, an audio out, a video out, a remote control output, or any combination thereof. As shown in FIG. 16, the system 1600 may include a smart-film filter adjustment module 1650 operable to adjusts the filter properties of a smart-film filter of the tanning apparatus (e.g., using a touch screen of a tablet) in accordance with the present disclosure, a smart base rotating motor 1670 operable to rotate a support connected to a smart base of the tanning apparatus (e.g., using a touch screen of a tablet) in accordance with the present disclosure, a smart-film filter controller 926 operable to control a smart-film filter in accordance with the present disclosure, a smart base controller 1026 operable to control the smart base in accordance with the present disclosure, a smart-film filter sensor 925 operable to receive and send signals to the smart-film filter of the tanning apparatus in accordance with the present disclosure, a smart base sensor 1025 operable to receive and send signals to the smart base of the tanning apparatus in accordance with the present disclosure, and a smart base pivoting arrangement 111 operable to tilt or adjust the angle of a mount of the tanning apparatus in accordance with the present disclosure.

Furthermore, the aspects of the disclosure may take the form of a computer program product accessible from a computer-usable or computer-readable medium providing program code for use by or in connection with a computer or any instruction execution system. The software and/or computer program product can be implemented in the environment of FIG. 16. For the purposes of this description, a computer-usable or computer readable medium can be any apparatus that can contain, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. Examples of a computer-readable storage medium include a semiconductor or solid state memory, magnetic tape, a removable computer diskette, a random access memory (RAM), a read-only memory (ROM), a rigid magnetic disk and an optical disk. Current examples of optical disks include compact disk-read only memory (CD-ROM), compact disc-read/write (CD-R/W) and DVD.

Although the present specification describes components and functions that may be implemented in particular embodiments with reference to particular standards and protocols (e.g., MIDI, pads), the disclosure is not limited to such standards and protocols. Such standards are periodically superseded by faster or more efficient equivalents having essentially the same functions. Accordingly, replacement standards and protocols having the same or similar functions are considered equivalents thereof.

While the computer-readable medium may be described as a single medium, the term “computer-readable medium” includes a single medium or multiple media, such as a centralized or distributed database, and/or associated caches and servers that store one or more sets of instructions. The term “computer-readable medium” shall also include any medium that is capable of storing, encoding or carrying a set of instructions for execution by a processor or that cause a computer system to perform any one or more of the embodiments disclosed herein.

The computer-readable medium may comprise a non-transitory computer-readable medium or media and/or comprise a transitory computer-readable medium or media. In a particular non-limiting, exemplary embodiment, the computer-readable medium can include a solid-state memory such as a memory card or other package that houses one or more non-volatile read-only memories. Further, the computer-readable medium can be a random access memory or other volatile re-writable memory. Additionally, the computer-readable medium can include a magneto-optical or optical medium, such as a disk, tapes or other storage device to capture carrier wave signals such as a signal communicated over a transmission medium. Accordingly, the disclosure is considered to include any computer-readable medium or other equivalents and successor media, in which data or instructions may be stored.

The illustrations of the embodiments described herein are intended to provide a general understanding of the structure of the various embodiments. The illustrations are not intended to serve as a complete description of all of the elements and features of apparatus and systems that utilize the structures or methods described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

One or more embodiments of the disclosure may be referred to herein, individually and/or collectively, by the term “invention” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

Unless otherwise defined, all technical and scientific terms used herein have the same meaning as commonly understood by one of ordinary skill in the art to which this disclosure belongs. The terminology used in the description of the disclosure herein is for describing particular embodiments only and is not intended to be limiting of the disclosure. As used in the description of the disclosure and the appended claims, the singular forms “a,” “an,” and “the” are intended to include the plural forms as well, unless the context clearly indicates otherwise.

Unless otherwise indicated, all numbers expressing quantities of ingredients and the like used in the specification and claims are to be understood as being modified in all instances by the term “about.” The terms “generally” and “approximately” as used herein are to be understood as an approximation allowing for variations while still maintaining the efficacy of the disclosure. Accordingly, unless indicated to the contrary, the numerical and geometric parameters and relationships set forth in the following specification and attached claims are approximations that may vary depending upon the desired properties sought to be obtained by the present disclosure. At the very least, and not as an attempt to limit the application of the doctrine of equivalents to the scope of the claims, each numerical and geometric parameters and relationship should be construed in light of the number of significant digits and ordinary rounding approaches.

The Abstract of the Disclosure is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

The above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description.

What is claimed is:

1. A tanning apparatus comprising:
a support having a bottom and a top;
a mount connected to the top of the support and configured to extend laterally from the support; and
at least one light filter, wherein
the mount is operable to releasably hold the at least one light filter,
the mount is an adjustably-sizable mount, and
the adjustably-sizable mount is operable to accommodate the at least one light filter.

2. The tanning apparatus of claim 1, wherein the adjustably-sizable mount comprises a longitudinally-extending frame connected to the top of the support and extending in a longitudinal direction, the frame having a first end and a second end.

3. The tanning apparatus of claim 2, wherein the adjustably-sizable mount further comprises at least one arm connected to and extending from at least one end of the longitudinally-extending frame in a transverse direction approximately perpendicular to the longitudinal direction.

4. The tanning apparatus of claim 3, wherein the at least one arm is configured to increase in overall length in the transverse direction to accommodate at least one additional light filter.

5. The tanning apparatus of claim 3, wherein the adjustably-sizable mount further comprises a second longitudinally-extending frame connected to the at least one arm and extending in the longitudinal direction.

6. The tanning apparatus of claim 2, wherein the longitudinally-extending frame is configured to increase in overall length in the longitudinal direction to accommodate at least one additional light filter.

7. The tanning apparatus of claim 1, wherein the adjustably-sizable mount includes at least one telescoping member.

8. The tanning apparatus of claim 1, wherein the adjustably-sizable mount includes at least one folding member.

9. The tanning apparatus of claim 1, wherein the adjustably-sizable mount includes at least two separate pieces assembleable together.

10. The tanning apparatus of claim 1, further comprising a joint arranged between the mount and the support, the joint enabling a relative pivoting movement between the mount and the support.

11. The tanning apparatus of claim 1, wherein the support is a single support extending from the bottom to the top in an approximately vertical direction.

12. The tanning apparatus of claim 1, wherein the support is configured to be removably embedded in ground.

13. The tanning apparatus of claim 1, further comprising a base, wherein the support is configured to be removably attached to the base.

14. The tanning apparatus of claim 13, further comprising a controller, wherein the base is operable to rotate the support, and the controller is configured to control rotation of the support.

15. The tanning apparatus of claim 14, further comprising a sensor, wherein the controller is configured to control rotation of the support in response to a detection by the sensor.

16. The tanning apparatus of claim 1, further comprising a controller, wherein the support is operable to rotate the mount, and the controller is configured to control position of the mount.

17. The tanning apparatus of claim 1, wherein the at least one filter comprises a plurality of filters supported along the mount, wherein at least two of the plurality of filters have different filtering properties from each other.

18. The tanning apparatus of claim 1, wherein the mount is generally rectangular.

19. A tanning apparatus comprising:
a support having a bottom and a top;
a mount connected to the top of the support and configured to extend laterally from the support; and
a plurality of light filters, wherein
the mount is operable to releasably hold the plurality of light filters, and
at least two of the plurality of light filters have different filtering properties from each other.

20. The tanning apparatus of claim 19, further comprising a plurality of joints, wherein each of the plurality of joints is configured to selectively connect:
two of the plurality of light filters to each other, or one of the plurality of light filters to the mount.

21. The tanning apparatus of claim 19, wherein the support comprises a telescoping pole arrangeable in an approximately vertical direction and configured to selectively extend and retract in the approximately vertical direction.

22. The tanning apparatus of claim 19, wherein the plurality of light filters comprise a user-selectable film tinting actuable by a controller configured to control the user-selectable film tinting based on a user selection.

23. The tanning apparatus of claim 19, wherein the plurality of light filters are at least one of a planar filter or a roll filter, and the mount is configured to support each of the planar filter and the roll filter.

24. The tanning apparatus of claim 23, wherein the mount is configured to concurrently support each of the planar filter and the roll filter.

25. The tanning apparatus of claim 19, wherein the plurality of light filters comprise at least one roll filter, wherein the roll filter in an un-deployed orientation is unrollable to position the roll filter on the mount in a deployed orientation.

26. The tanning apparatus of claim 25, wherein the roll filter comprises different filter regions along a length of the roll filter, at least two of the different filter regions having differing light filtering properties from each other.

27. The tanning apparatus of claim 25, further comprising a second roll filter, wherein the roll filter in the un-deployed orientation is supportable on a first end of the mount, and the second roll filter in the un-deployed orientation is supportable on a second end of the mount.

28. The tanning apparatus of claim 19, wherein the plurality of light filters comprise at least one of glass and/or plastic.

29. The tanning apparatus of claim 19, wherein at least one of the plurality of light filters is a photochromatic filter.

30. The tanning apparatus of claim 19, wherein at least one of the plurality of light filters is an electrochromatic filter.

31. The tanning apparatus of claim 19, wherein at least one of the plurality of light filters is a filter having fixed filtering properties.

32. The tanning apparatus of claim 19, wherein the plurality of light filters are planar filters.

33. The tanning apparatus of claim 32, wherein the plurality of light filters are rigid filters.

34. The tanning apparatus of claim 19, wherein the plurality of light filters comprise at least three filters configured to be arranged serially along a longitudinal direction of the mount, wherein at least two of the at least three filters have different filtering properties from each other.

35. A tanning apparatus comprising:
a support having a bottom and a top;
a base releasably attachable at the bottom of the support;
a mount connected to the top of the support and configured to extend laterally from the support; and
at least one light filter, wherein
the mount is operable to releasably hold the at least one light filter,
the mount is configured to adjustably pivot relative to the support,
the support can vertically adjust.

36. The tanning apparatus of claim 35, wherein the support is attached at an approximately mid-way point along the mount.
